(19) United States
(12) Patent Application Publication
(10) Pub. No.: US 2026/0279524 A1
(43) Pub. Date: Sep. 17, 2026

(54) INFORMATION PROCESSING APPARATUS, INFORMATION PROCESSING METHOD, AND NON-TRANSITORY COMPUTER-READABLE MEDIUM

(71) Applicants: NEC Corporation, Tokyo (JP); SOCIAL WELFARE ORGANIZATION SAISEIKAI IMPERIAL GIFT FOUNDATION, INC. SAISEIKAI KUMAMOTO HOSPITAL, Kumamoto-shi (JP)

(72) Inventors: Kei SHIBUYA, Tokyo (JP); Konosuke TEMMEI, Tokyo (JP); Shuhei NOYORI, Tokyo (JP); Mieko MANO, Tokyo (JP); Keiichirou OKAMOTO, Tokyo (JP); Sumihiro SHIRAI, Kumamoto (JP); Hideyuki TANAKA, Kumamoto (JP); Shinji TAKAMUNE, Kumamoto (JP); Rui SUGETA, Kumamoto (JP)

(73) Assignees: NEC Corporation, Tokyo (JP); SOCIAL WELFARE ORGANIZATION SAISEIKAI IMPERIAL GIFT FOUNDATION, INC.SAISEIKAI KUMAMOTO HOSPITAL, Kumamoto-shi (JP)

(21) Appl. No.: 19/564,701

(22) Filed: Mar. 12, 2026

(30) Foreign Application Priority Data
Mar. 14, 2025 (JP) 2025-040925

(51) Int. Cl. G16H 10/60; G16H 50/30

(52) U.S. Cl. CPC G16H 10/60; G16H 50/30

(57) ABSTRACT

An object is to provide an information processing apparatus capable of easily collecting data necessary for risk assessment of surgery. An information processing apparatus according to the present disclosure includes a patient data acquisition unit that acquires patient data that is extracted from an electronic medical record and indicates a condition of a patient, a necessary input item acquisition unit that acquires an input item necessary for risk assessment for surgery of the patient, a necessary data collection unit that collects patient data necessary for the risk assessment based on the necessary input item and the patient data, and a risk assessment request unit that requests the risk assessment by using the collected patient data.

### INCORPORATION BY REFERENCE

This application is based upon and claims the benefit of priority from Japanese patent application No. 2025-040925, filed on Mar. 14, 2025, the disclosure of which is incorporated herein in its entirety by reference.

### TECHNICAL FIELD

The present disclosure relates to an information processing apparatus, an information processing method, and a program.

### BACKGROUND ART

A technique for assessing a risk of surgery performed on a patient in a medical institution is known. As a related art, JP 2018-532114 A discloses a method for predicting the risk of a patient suffering from acute kidney injury (AKI) during or after a surgical procedure or after administration of a contrast agent.

### SUMMARY

Pre-surgical risk assessment for a patient is important for both doctors and patients. In order to appropriately perform the risk assessment, it is necessary to collect various types of data that may affect the risk of surgery, such as a blood pressure, a heart rate, or a blood glucose level of the patient, for example. However, these types of data are scattered on an electronic medical record, and it may be difficult to obtain these types of data at a time. A doctor or a medical worker needs to input items necessary for risk assessment on a predetermined input screen while searching for necessary data on an electronic medical record, and thus a burden is caused in performing risk assessment.

In view of the above-described problems, an example object of the present disclosure is to provide an information processing apparatus, an information processing method, and a program capable of easily collecting data necessary for risk assessment of surgery.

An information processing apparatus according to an example aspect of the present disclosure includes
a patient data acquisition unit that acquires patient data that is extracted from an electronic medical record and indicates a condition of a patient,
a necessary input item acquisition unit that acquires an input item necessary for risk assessment for surgery of the patient,
a necessary data collection unit that collects patient data necessary for the risk assessment based on the necessary input item and the patient data, and
a risk assessment request unit that requests the risk assessment by using the collected patient data.

An information processing method according to another example aspect of the present disclosure includes
a patient data acquisition step for acquiring patient data that is extracted from an electronic medical record and indicates a condition of a patient,
a necessary input item acquisition step for acquiring an input item necessary for risk assessment for surgery of the patient,
a necessary data collection step for collecting patient data necessary for the risk assessment based on the necessary input item and the patient data, and
a risk assessment request step for requesting the risk assessment by using the collected patient data.

A program according to still another example aspect of the present disclosure causes a computer to execute
a patient data acquisition step for acquiring patient data that is extracted from an electronic medical record and indicates a condition of a patient,
a necessary input item acquisition step for acquiring an input item necessary for risk assessment for surgery of the patient,
a necessary data collection step for collecting patient data necessary for the risk assessment based on the necessary input item and the patient data, and
a risk assessment request step for requesting the risk assessment by using the collected patient data.

The information processing apparatus, the information processing method, and the program according to the example aspects of the present disclosure have an example effect of enabling easy collection of data necessary for risk assessment of surgery.

### BRIEF DESCRIPTION OF DRAWINGS

The above and other aspects, features and advantages of the present disclosure will become more apparent from the following description of certain exemplary embodiments when taken in conjunction with the accompanying drawings, in which:

FIG. 1 is a block diagram illustrating a configuration of an information processing apparatus according to the present disclosure;

FIG. 2 is a flowchart illustrating a flow of processing performed by the information processing apparatus;

FIG. 3 is a diagram illustrating a relationship between an input of a pre-surgical risk of a patient and calculation of a predicted value of a post-surgical outcome;

FIG. 4 is a block diagram illustrating a configuration of an information processing system according to the present disclosure;

FIG. 5 is a diagram illustrating an example of a correspondence table used in collection of patient data in a necessary data collection unit;

FIG. 6 is a view illustrating an example of a display screen for displaying an assessment result of a pre-surgical risk in the same assessment index;

FIG. 7 is a view illustrating an example of a display screen for displaying an assessment result of a pre-surgical risk in a plurality of assessment indices;

FIG. 8 is a view illustrating an example of a display screen for further displaying a proposal to a doctor;

FIG. 9 is a flowchart illustrating a flow of processing performed by a server;

FIG. 10 is a block diagram illustrating a configuration of a processing block according to a modified example of a second example embodiment; and

FIG. 11 is a block diagram exemplifying a hardware configuration of a computer that implements the information processing apparatus and the like.

### EXAMPLE EMBODIMENTS

Hereinafter, example embodiments of the present disclosure will be described in detail with reference to the drawings. In the drawings, the same or relevant elements are denoted by the same reference signs. For clarity of description, redundant description will be omitted as necessary.

### First Example Embodiment

### (Information Processing Apparatus 1)

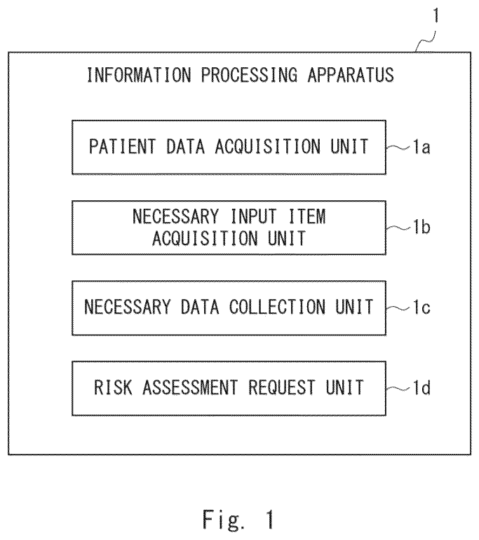

FIG. 1 is a block diagram illustrating a configuration of an information processing apparatus 1 according to the present disclosure. The information processing apparatus 1 includes a patient data acquisition unit 1a, a necessary input item acquisition unit 1b, a necessary data collection unit 1c, and a risk assessment request unit 1d.

The patient data acquisition unit 1a acquires patient data that is extracted from an electronic medical record and indicates the condition of a patient. The necessary input item acquisition unit 1b acquires an input item necessary for risk assessment for surgery of the patient. The necessary data collection unit 1c collects patient data necessary for risk assessment based on a necessary input item and patient data. The risk assessment request unit 1d requests risk assessment by using the collected patient data.

### (Processing of Information Processing Apparatus 1)

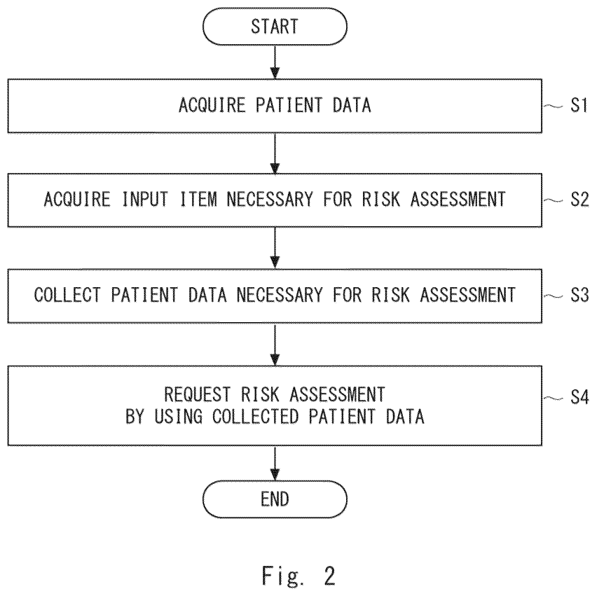

Next, processing performed by the information processing apparatus 1 will be described with reference to FIG. 2. FIG. 2 is a flowchart illustrating a flow of the processing performed by the information processing apparatus 1.

First, the patient data acquisition unit 1a acquires patient data that is extracted from an electronic medical record and indicates the condition of a patient (S1). Then, the necessary input item acquisition unit 1b acquires an input item necessary for risk assessment for surgery of the patient (S2). Subsequently, the necessary data collection unit 1c collects patient data necessary for risk assessment based on a necessary input item and patient data (S3). The risk assessment request unit 1d requests risk assessment by using the collected patient data (S4).

As described above, according to the information processing apparatus 1 of the present disclosure, patient data necessary for risk assessment is collected based on a necessary input item and patient data, and thus, it is possible to easily collect data necessary for risk assessment of surgery.

### Second Example Embodiment

Subsequently, a second example embodiment will be described. A second example embodiment is a specific example of the first example embodiment described above. First, positioning of the present disclosure will be briefly described. The present disclosure contributes to reduction in burden of work on a doctor and improvement in quality of medical care. For example, the present disclosure is in response to the “working-style revolution for doctors” started in 2024, and aims to maintain or improve a health care balance in hospitals and the like while reducing working hours of doctors.

In particular, the present disclosure focuses on support in assessing a risk that occurs post-surgery based on a pre-surgical condition of a patient. The present disclosure contributes to change of a treatment policy, improvement of a post-surgical outcome, or improvement of a hospital bed rotation rate, by accurately assessing a risk associated with surgery of a patient in advance. As a result, the present disclosure enables optimization of medical resources while reducing a burden on a doctor.

Assessment of a risk occurring after surgery based on a condition of a patient before surgery is referred to as “pre-surgical risk assessment” or simply “risk assessment” below. A risk as an assessment target in the pre-surgical risk assessment is referred to as “pre-surgical risk” or simply “risk”.

As described above, in the related technique, it is necessary to collect patient data scattered in electronic medical records for pre-surgical risk assessment, and it takes a lot of time for a doctor to manually search and input information. The present disclosure automatically extracts necessary information from an electronic medical record, assists input for risk assessment, and supports comparison and visualization of results.

Furthermore, the present disclosure enables adaptation determination of surgery and early identification of a patient with a high risk, by utilizing artificial intelligence (AI) and performing prediction support of a pre-surgical risk. As a result, a doctor can make a more accurate determination and achieve efficient hospital management while maintaining the quality of medical care.

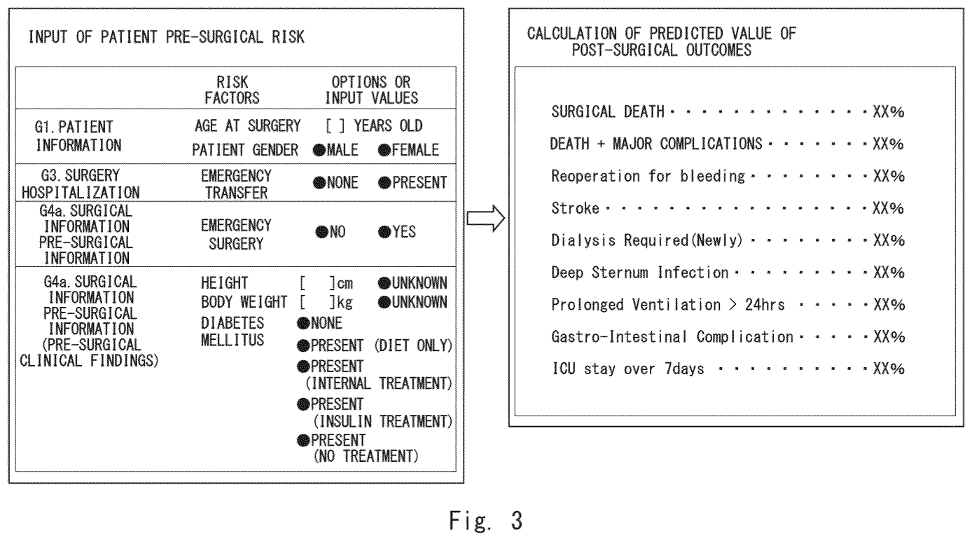

Subsequently, problems according to the present disclosure will be described more specifically with reference to FIG. 3. FIG. 3 is a diagram illustrating a relationship between an input of a pre-surgical risk of a patient and calculation of a predicted value of a post-surgical outcome. On the left side of FIG. 3, input items of patient information necessary for pre-surgical risk assessment, such as a surgical age, a sex, presence or absence of emergency transport, implementation of emergency surgery, a height, a body weight, and presence or absence of diabetes mellitus are illustrated. These types of information are recorded by selecting appropriate risk factors from among options or inputting numerical values. These types of information may be included in text information such as a doctor record and a nursing record.

On the right side of FIG. 3, a predicted value of a post-surgical outcome calculated based on the input information is illustrated. Specifically, a surgical mortality rate, an incidence rate of major complications, a risk of re-surgery due to bleeding, an incidence rate of stroke, a need for new dialysis, a risk of deep sternal infection, a need for mechanical ventilation management, an incidence rate of gastrointestinal complications, a probability of an intensive care unit (ICU) stay period exceeding 7 days, and the like are illustrated as numerical values. As a result, a doctor can ascertain the pre-surgical risk of the patient in detail, and can examine an appropriate surgical plan and post-surgical management.

However, in a medical institution, input data as illustrated on the left side may be scattered in a plurality of data sources including electronic medical records. Therefore, in order to collect such input data, a doctor or a medical worker needs to collect information necessary for pre-surgical risk assessment from various data sources. A burden on manual data collection and input work by a doctor or a medical worker is large, and there is a problem that it is difficult to perform pre-surgical risk assessment in real time. The present disclosure solves such a problem.

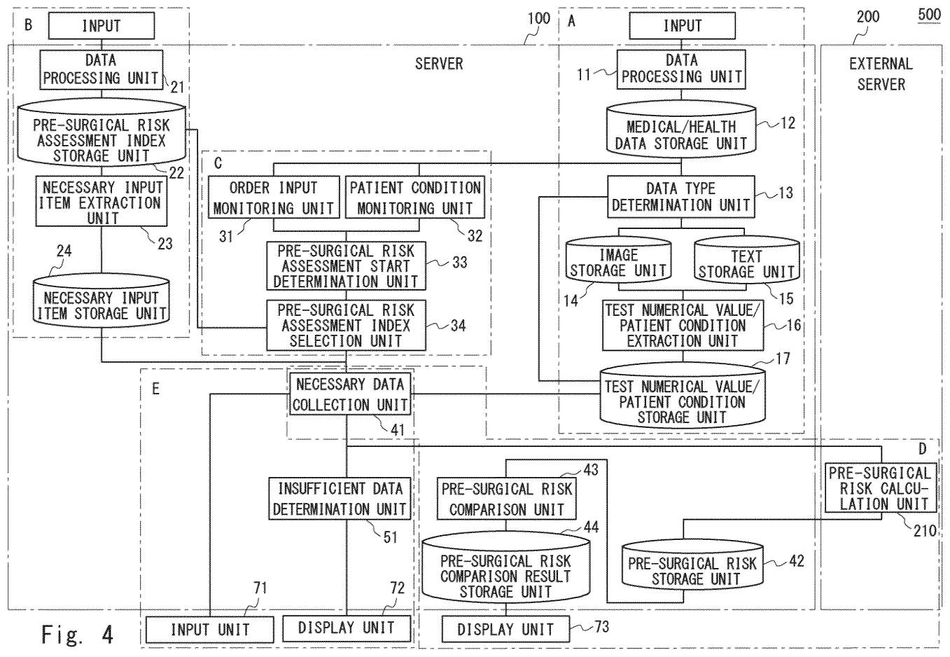

FIG. 4 is a block diagram illustrating a configuration of an information processing system 500 according to the present disclosure. An information processing system 500 is an information processing system that collects patient data scattered in an electronic medical record and extracts information necessary for pre-surgical risk assessment. The information processing system 500 predicts a pre-surgical risk indicating a success rate of surgery and a risk of complications based on patient data such as the age, a medical history, a test result, and a general condition, for example. The pre-surgical risk indicates a risk of affecting complications related to a patient undergoing surgery and progress after the surgery.

The information processing system 500 includes a server 100 and an external server 200. The server 100 is an example of the information processing apparatus 1 described above. The server 100 is, for example, an in-hospital server provided in a hospital. The external server 200 is, for example, an out-of-hospital server provided in an external network of a medical institution. The server 100 and the external server 200 are connected via a network and transmit and receive data to and from each other. The type of communication may be wired communication or wireless communication.

The server 100 is an information processing apparatus that acquires patient data from an electronic medical record, performs predetermined processing necessary for pre-surgical risk assessment, and requests the external server 200 for pre-surgical risk assessment. The external server 200 is an information processing apparatus that receives a request from the server 100, performs pre-surgical risk assessment, and transmits an assessment result as a response to the server 100.

### [Server 100]

First, the server 100 will be described. The server 100 acquires patient data from an electronic medical record and performs predetermined processing necessary for pre-surgical risk assessment. A configuration of the server 100 will be described below separately for processing blocks A to E illustrated in FIG. 4.

### (Processing Block A)

The processing block A is a block that processes data input to the server 100 and performs processing of classifying, extracting, and storing data necessary for pre-surgical risk assessment. The processing block A functions as an electronic medical record data organization unit that organizes electronic medical record data included in an electronic medical record for use in risk assessment. The processing block A determines whether the electronic medical record data is directly available for risk assessment based on a data type of the electronic medical record data, and performs predetermined processing in a case where it is determined that the electronic medical record data is not directly available.

The processing block A includes a data processing unit 11, a medical/health data storage unit 12, a data type determination unit 13, an image storage unit 14, a text storage unit 15, a test numerical value/patient condition extraction unit 16, and a test numerical value/patient condition storage unit 17.

First, the data processing unit 11 acquires patient data input by typing, voice input, sensing, or the like. The data processing unit 11 receives an input of patient data from a doctor or a medical worker via an electronic medical record. A doctor or a medical worker inputs patient data on an electronic medical record by using a terminal device such as a personal computer (PC) or a smartphone, for example.

Patient data is data regarding a patient and includes data that may affect a risk of surgery. The patient data includes, for example, a height, a body weight, a test value, a medical history, a clinical history, and the like. These are examples, and patient data may include various types of data that may affect the risk. For example, the patient data may include a patient age, a sex, known pre-surgical risk information, and the like.

The data processing unit 11 processes input patient data and converts the patient data into a format that can be stored in the medical/health data storage unit 12. The data processing unit 11 stores the converted patient data in the medical/health data storage unit 12.

The medical/health data storage unit 12 stores patient data regarding the medical care and health of a patient, such as an electronic medical record and a life log. The medical/health data storage unit 12 stores, for example, a patient ID for identifying a patient and patient data of the patient in association with each other. The patient ID may be commonly used in storage units other than the medical/health data storage unit 12. The medical/health data storage unit 12 sends the data to an order input monitoring unit 31 and a patient condition monitoring unit 32 in the processing block C.

The data type determination unit 13 determines whether data stored in the medical/health data storage unit 12 can be directly used in subsequent processing or whether additional processing is necessary. The data that can be directly used is, for example, quantitative measurement data. The quantitative measurement data is, for example, data recorded in a numerical value, a score, a classification format, or the like. The quantitative measurement data includes, for example, a test value, a vital sign, and the like.

The test value is numerical data obtained as a result of a blood test, a urine test, or the like. The test value is, for example, a blood glucose level, HbA1c, a renal function, a liver function, an electrolyte, the number of white blood cells, the number of red blood cells, the number of platelets, or the like. The vital sign is an index indicating a physiological condition of the patient. The vital sign is, for example, blood pressure, a heart rate, a body temperature, a respiratory rate, an oxygen saturation, or the like.

For example, the data type determination unit 13 analyzes a data structure in the patient data and specifies a field in which the test value and the vital sign are stored. The data type determination unit 13 determines whether data in the specified field is quantitative measurement data such as the test value and the vital sign. In a case of determining that the data is quantitative measurement data, the data type determination unit 13 determines that the data is directly available in subsequent processing.

The data type determination unit 13 may determine whether a format, a numerical value, a unit, or the like of the patient data is appropriate by comparison with a reference value. For example, in a case where the patient data is quantitative measurement data and the patient data is in an appropriate format, the data type determination unit 13 determines that the patient data is directly available. In a case of determining that the patient data is directly available, the data type determination unit 13 sends the data to the test numerical value/patient condition storage unit 17.

In a case of determining that the patient data is not directly available, the data type determination unit 13 sends the data to the image storage unit 14 or the text storage unit 15. Specifically, in a case where the patient data is image data, the data type determination unit 13 sends the patient data to the image storage unit 14. In a case where the patient data is text data, the data type determination unit 13 sends the patient data to the text storage unit 15.

The image storage unit 14 stores image data acquired from the data type determination unit 13. The text storage unit 15 stores the text data acquired from the data type determination unit 13.

The test numerical value/patient condition extraction unit 16 extracts information regarding the test numerical value and the patient condition from image data and text data that are patient data. The information regarding the test numerical value and the patient condition is, for example, information such as the test value and the vital sign. For example, the test numerical value/patient condition extraction unit 16 extracts the body temperature and the presence or absence of smoking from a text recorded at time of interview. The test numerical value/patient condition extraction unit 16 sends the extracted test numerical value and the like to the test numerical value/patient condition storage unit 17. In this manner, the test numerical value/patient condition extraction unit 16 can convert patient data of the data type determined not to be directly available into a format that can be used in the subsequent processing.

The test numerical value/patient condition storage unit 17 stores information of the extracted test numerical value and patient condition. As a result, these pieces of information can be used in subsequent processing.

### (Processing Block B)

The processing block B is a block that manages information of a known assessment index, extracts an input item necessary for pre-surgical risk assessment, and performs processing of storing the input item. The assessment index serves as a reference for quantitatively assessing a risk associated with surgery. The processing block B functions as a necessary input item management unit that manages a necessary input item based on an assessment index set in advance as a reference of risk assessment. The processing block B includes a data processing unit 21, a pre-surgical risk assessment index storage unit 22, a necessary input item extraction unit 23, and a necessary input item storage unit 24.

The data processing unit 21 processes the input index data and stores the index data in an appropriate format. The index data includes information of a known assessment index. In a case where a new assessment index is created, for example, a doctor or a medical worker inputs the content of the assessment index to the server 100. In a case where index data including a new assessment index is input, the data processing unit 21 processes the new index data. For example, in a case where a PDF file is input as index data, the data processing unit 21 extracts text data indicating an assessment index from the PDF file. The data processing unit 21 stores the extracted text data in the pre-surgical risk assessment index storage unit 22.

The pre-surgical risk assessment index storage unit 22 stores an assessment index. For example, the pre-surgical risk assessment index storage unit 22 manages surgery performed on a patient and an assessment index in association with each other. The pre-surgical risk assessment index storage unit 22 may store a plurality of assessment indices for one surgery. In a case where a new assessment index is added, the pre-surgical risk assessment index storage unit 22 updates the content of the pre-surgical risk assessment index storage unit 22.

The necessary input item extraction unit 23 extracts an input item necessary for risk assessment from the pre-surgical risk assessment index storage unit 22, and stores an extraction result in the necessary input item storage unit 24. For example, the necessary input item extraction unit 23 extracts an age, a sex, a height, a body weight, a diabetes mellitus history, or the like as illustrated in FIG. 3 as input items necessary for risk assessment.

The necessary input item extraction unit 23 sends the extracted necessary input items to the necessary input item storage unit 24. The necessary input item storage unit 24 stores necessary input items received from the necessary input item extraction unit 23.

### (Processing Block C)

The processing block C is a block that monitors a change in a surgery order or a patient condition and selects a start timing of the pre-surgical risk assessment and an appropriate assessment index. The processing block C functions as a monitoring unit that monitors at least one of an order status of surgery to be performed on a patient and a condition of the patient and determines whether to perform risk assessment based on a monitoring result. The processing block C determines to perform risk assessment in at least one of a case where surgery of a patient is ordered and a case where an abnormality is detected in the condition of the patient.

The processing block C includes an order input monitoring unit 31, a patient condition monitoring unit 32, a pre-surgical risk assessment start determination unit 33, and a pre-surgical risk assessment index selection unit 34.

The order input monitoring unit 31 monitors medical instructions such as a surgery order and a test order on an electronic medical record, and determines whether an instruction related to surgery has been issued. The order input monitoring unit 31 determines that an instruction related to surgery has been issued in a case where determination of surgery, start of pre-surgical preparation, or the like is detected in data on an electronic medical record. The order input monitoring unit 31 sends a determination result to the pre-surgical risk assessment start determination unit 33. For example, in a case where it is determined that the instruction related to the surgery has been issued, the order input monitoring unit 31 transmits a patient ID and information regarding the surgery to the pre-surgical risk assessment start determination unit 33.

The patient condition monitoring unit 32 monitors a test value and biological information acquired from an electronic medical record, a vital monitor, or the like, and detects a change in the condition of a patient. The patient condition monitoring unit 32 monitors a change in the test value such as blood pressure, a heart rate, a blood glucose level, or the like. The patient condition monitoring unit 32 determines whether an abnormality has occurred in these test values. The patient condition monitoring unit 32 sends a determination result to the pre-surgical risk assessment start determination unit 33. For example, in a case where it is determined that an abnormality has occurred in these test values, the patient condition monitoring unit 32 sends the patient ID and information regarding the occurred abnormality to the pre-surgical risk assessment start determination unit 33.

The pre-surgical risk assessment start determination unit 33 determines whether to start the pre-surgical risk assessment. In a case where the order input monitoring unit 31 determines that an instruction related to surgery has been issued, the pre-surgical risk assessment start determination unit 33 determines to start the pre-surgical risk assessment. The pre-surgical risk assessment start determination unit 33 also determines to start the pre-surgical risk assessment in a case where the patient condition monitoring unit 32 determines that an abnormality has occurred in the condition of the patient.

The pre-surgical risk assessment start determination unit 33 may determine to start the pre-surgical risk assessment in response to a request from a doctor at any timing. For example, in a case where a doctor presses a “pre-surgical risk assessment start button” provided on a display screen on an electronic medical record or the accompanying option, the pre-surgical risk assessment start determination unit 33 receives a request for starting the pre-surgical risk assessment from the doctor. In a case where it is determined to start the pre-surgical risk assessment, the pre-surgical risk assessment start determination unit 33 sends a message indicating the start of the pre-surgical risk assessment to the pre-surgical risk assessment index selection unit 34. For example, the pre-surgical risk assessment start determination unit 33 sends the patient ID, and information regarding surgery or information regarding an abnormality that has occurred in the patient to the pre-surgical risk assessment index selection unit 34.

In a case where the pre-surgical risk assessment start determination unit 33 determines to start the pre-surgical risk assessment, the pre-surgical risk assessment index selection unit 34 selects an assessment index suitable for the case of the patient. For example, the pre-surgical risk assessment index selection unit 34 refers to the pre-surgical risk assessment index storage unit 22, and selects an assessment index suitable for this case from a plurality of assessment indices stored. The assessment index suitable for this case is an assessment index associated with the surgery for which the instruction has been issued.

The present disclosure is not limited to this, and the pre-surgical risk assessment index selection unit 34 may select the assessment index by selection of the doctor. For example, the doctor selects an assessment index to be used on a predetermined display screen. The pre-surgical risk assessment index selection unit 34 receives this selection and selects an assessment index to be used for the patient. The pre-surgical risk assessment index selection unit 34 sends the patient ID and the selected assessment index to the necessary data collection unit 41.

### (Processing Block D)

The processing block D is a block that performs processing of collecting data necessary for pre-surgical risk assessment, assessing a pre-surgical risk at a plurality of timings, and presenting, to a doctor, a result of comparing the assessments at the plurality of timings. The processing block D includes a necessary data collection unit 41, a pre-surgical risk storage unit 42, a pre-surgical risk comparison unit 43, a pre-surgical risk comparison result storage unit 44, a display unit 73, and a pre-surgical risk calculation unit 210 of the external server 200.

The necessary data collection unit 41 is an example of the patient data acquisition unit 1a, the necessary input item acquisition unit 1b, the necessary data collection unit 1c, and the risk assessment request unit 1d, which have been described above. The necessary data collection unit 41 functions as the patient data acquisition unit 1a that acquires patient data that is extracted from an electronic medical record and indicates the condition of a patient. The necessary data collection unit 41 functions as the necessary input item acquisition unit 1b that acquires an input item necessary for risk assessment for surgery of the patient. Furthermore, the necessary data collection unit 41 functions as the necessary data collection unit 1c that collects patient data necessary for risk assessment based on the necessary input item and the patient data. The necessary data collection unit 41 functions as the risk assessment request unit 1d that requests risk assessment by using the collected patient data.

For example, the necessary data collection unit 41 (patient data acquisition unit 1a) acquires patient data included in the electronic medical record data organized in the processing block A. The necessary data collection unit 41 (necessary input item acquisition unit 1b) acquires a necessary input item based on the assessment index managed in the processing block B.

In a case where it is determined that the risk assessment is to be performed in the processing block C (monitoring unit), the necessary data collection unit 41 (necessary data collection unit 1c) starts collecting patient data necessary for the risk assessment. The necessary data collection unit 41 may automatically start collecting the patient data. Specifically, the necessary data collection unit 41 (necessary data collection unit 1c) collects necessary patient data based on a predetermined correspondence table in which necessary input items and patient data including descriptions relevant to the necessary input items are associated with each other. The necessary data collection unit 41 (risk assessment request unit 1d) requests the external server 200 to execute risk assessment by using the collected patient data.

The processing of the necessary data collection unit 41 will be specifically described. The necessary data collection unit 41 collects patient data necessary for pre-surgical risk assessment based on a test numerical value and information of the patient condition. Specifically, the necessary data collection unit 41 refers to the correspondence table created based on the necessary input item storage unit 24 and the test numerical value/patient condition storage unit 17, and extracts appropriate patient data. The correspondence table is information in which an input item necessary for performing the pre-surgical risk assessment is associated with a document in which a test value of the input item and the like have been described. The necessary data collection unit 41 can collect patient data necessary for performing the pre-surgical risk assessment by referring to the correspondence table. The necessary data collection unit 41 may store the correspondence table in advance in a storage unit (not illustrated).

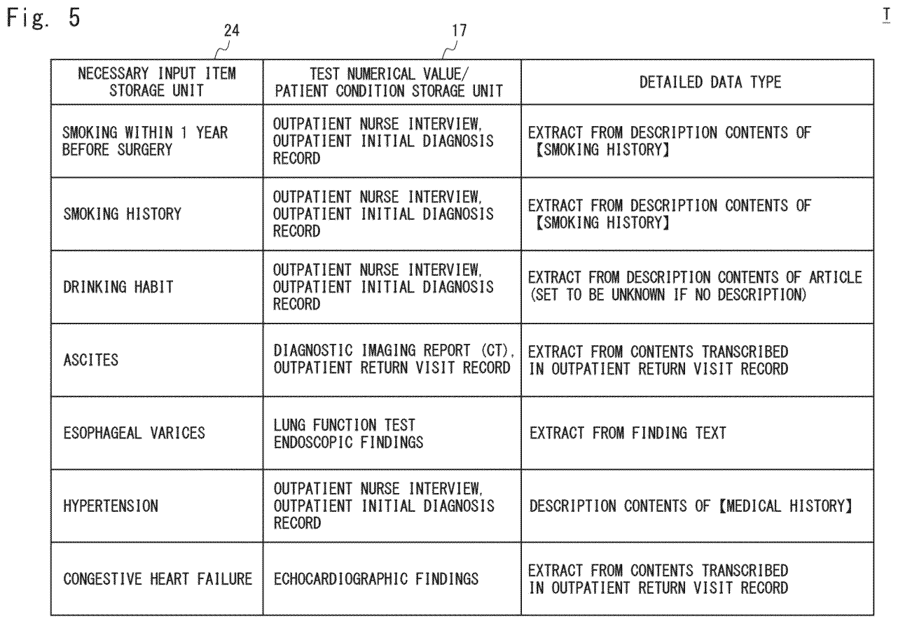

FIG. 5 is a diagram illustrating an example of a correspondence table T used in collection of patient data in the necessary data collection unit 41. As illustrated in FIG. 5, the correspondence table T is obtained by associating data stored in the necessary input item storage unit 24 with data stored in the test numerical value/patient condition storage unit 17. For example, the necessary data collection unit 41 extracts data regarding a smoking history of the patient from the description contents of “outpatient nurse interview” or “outpatient initial diagnosis record”. The necessary data collection unit 41 extracts data regarding esophageal varices from the description contents of the endoscopic findings of a “pulmonary function test”. The necessary data collection unit 41 collects the extracted data.

The description will return to FIG. 4. The necessary data collection unit 41 transmits the patient ID and the collected data to the pre-surgical risk calculation unit 210 of the external server 200. The necessary data collection unit 41 requests the pre-surgical risk calculation unit 210 to perform pre-surgical risk assessment.

The pre-surgical risk calculation unit 210 of the external server 200 performs the pre-surgical risk assessment in response to a request from the necessary data collection unit 41. The pre-surgical risk calculation unit 210 performs the pre-surgical risk assessment by using the data collected by the necessary data collection unit 41 and the assessment index stored in the pre-surgical risk assessment index storage unit 22. For example, the pre-surgical risk calculation unit 210 performs the pre-surgical risk assessment by calculating a predicted value of a post-surgical outcome. The predicted value of the post-surgical outcome can be calculated, for example, as a numerical value of the incidence rate of complications after surgery, a possibility of re-hospitalization, the duration of ICU stay, the number of days of hospitalization, or the mortality rate.

The present disclosure is not limited to this, and the pre-surgical risk calculation unit 210 may assess the pre-surgical risk in any manner. The pre-surgical risk calculation unit 210 sends the patient ID and an assessment result to the pre-surgical risk storage unit 42. In this manner, the server 100 can collect necessary data, and the external server 200 can perform the pre-surgical risk assessment.

Here, since the known index is used as the assessment index, an example in which the pre-surgical risk assessment is requested from the server 100 to the external server 200 is used, but the present disclosure is not limited to this. The server 100 may have the function of the pre-surgical risk calculation unit 210, and the pre-surgical risk assessment may be performed in the server 100.

The pre-surgical risk storage unit 42 stores the assessment result of a pre-surgical risk acquired from the pre-surgical risk calculation unit 210. For example, the pre-surgical risk storage unit 42 stores a patient ID, assessment date and time, an assessment index, and an assessment result in association with each other. In a case where a plurality of pre-surgical risk assessments are performed at different assessment timings for one patient, the pre-surgical risk storage unit 42 stores each assessment result in association with the patient ID, the assessment date and time, and the assessment index. As a result, the pre-surgical risk storage unit 42 manages the assessment results at a plurality of assessment timings as time-series data.

The pre-surgical risk comparison unit 43 compares a plurality of pre-surgical risks. For example, the pre-surgical risk comparison unit 43 compares time-series changes in the same assessment index for one patient. For example, the pre-surgical risk comparison unit 43 acquires time-series data of a pre-surgical risk of a patient P from the pre-surgical risk storage unit 42 for an assessment index X. For example, the pre-surgical risk comparison unit 43 acquires four pre-surgical risks having different assessment dates and times of the pre-surgical risks, and compares the four pre-surgical risks. For example, the pre-surgical risk comparison unit 43 calculates an increase or decrease in the predicted value of the post-surgical outcome as a comparison result. The pre-surgical risk comparison unit 43 sends the comparison result to the pre-surgical risk comparison result storage unit 44.

The pre-surgical risk comparison unit 43 may compare time-series changes in a plurality of assessment indices. For example, the pre-surgical risk comparison unit 43 acquires time-series data of a pre-surgical risk of a patient P from the pre-surgical risk storage unit 42 for assessment indices X and Y. The pre-surgical risk comparison unit 43 calculates an increase or decrease in the pre-surgical risk for each of the assessment indices X and Y. In this manner, the pre-surgical risk comparison unit 43 can calculate the pre-surgical risk by using the plurality of assessment indices, and thus can more accurately assess the pre-surgical risk.

Furthermore, the pre-surgical risk comparison unit 43 may make a proposal to the doctor based on the comparison result of the pre-surgical risk. For example, the pre-surgical risk comparison unit 43 may propose reviewing a surgical plan. For example, it is assumed that, as a result of the risk assessment, the pre-surgical risk increases based on a specific index. In this case, the pre-surgical risk comparison unit 43 may propose a change to an appropriate surgeon depending on the difficulty of the surgery and the condition of the patient. Alternatively, the pre-surgical risk comparison unit 43 may propose prolongation of the number of days of hospitalization after surgery or adjustment of hospital transfer. In this manner, the pre-surgical risk comparison unit 43 can provide an appropriate determination material based on the comparison result of the pre-surgical risk.

The pre-surgical risk comparison result storage unit 44 stores the comparison result of the pre-surgical risk in the time-series data for each of the same assessment index and the plurality of assessment indices.

The display unit 73 displays a result of the pre-surgical risk assessment. The display unit 73 displays, for example, the predicted value of the post-surgical outcome indicating the magnitude of the risk in the surgery. The display unit 73 may display results at a plurality of assessment timings as time-series data. The display unit 73 may display a result calculated based on the same assessment index set in advance by using the predicted value and a time-series graph of the predicted value. The display unit 73 may display each of a plurality of results calculated based on a plurality of assessment indices set in advance, on the same screen. The display unit 73 may further display a comment including a proposal related to a policy for a surgery, the proposal being created based on the assessment result, on the same screen.

The display unit 73 will be specifically described. The display unit 73 displays the comparison result of the pre-surgical risk stored in the pre-surgical risk comparison result storage unit 44. The display unit 73 is a display device such as a display. The display unit 73 may be a touch panel having a function of an input unit. For example, the display unit 73 visually displays a time-series change in the same assessment index. The display unit 73 may display the transition of the pre-surgical risk by using a graph, a chart, or the like. In this manner, the doctor can easily ascertain the transition of the risk.

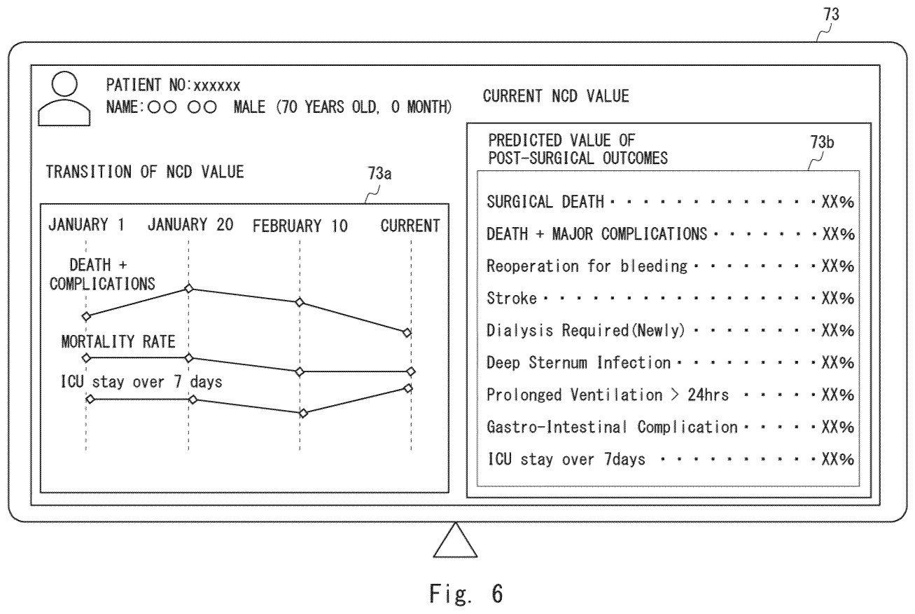

FIG. 6 is a view illustrating an example of a display screen for displaying the assessment result of the pre-surgical risk in the same assessment index. In the example of FIG. 6, the display unit 73 displays the assessment result of the pre-surgical risk using the National Clinical Database (NCD) as the assessment index. The display unit 73 displays a display region divided into left and right parts. For example, the display unit 73 displays a graph 73a showing a time-series change in NCD value (pre-surgical risk) on the left half part. The display unit 73 displays a predicted value 73b of a post-surgical outcome as the current value of the NCD value in the right half part.

The horizontal direction of the graph 73a indicates time, and the vertical direction indicates the probability (%) of the pre-surgical risk. The graph 73a visually shows a variation in pre-surgical risk over time. In this example, the graph 73a includes the probability of patient death or complication occurrence, the mortality rate, and the probability of the duration of ICU stay exceeding 7 days. In the graph 73a, the NCD value is calculated four times, that is, on January 1, January 20, February 10, and the current time. The graph 73a includes only three indices with high importance among the assessment indices.

The predicted value 73b includes a predicted value of the post-surgical outcome based on the current NCD value. For example, the predicted value 73b includes a plurality of predicted values, such as a mortality rate due to surgery, a risk of re-surgery due to bleeding, the incidence rate of stroke, and a need for mechanical ventilation management for more than 24 hours. As described above, the display unit 73 displays the graph 73a and the predicted value 73b on the same screen. As a result, the doctor can easily ascertain the pre-surgical risk by visually recognizing the display unit 73.

Since the example of FIG. 6 is just an example, the display unit 73 may display the pre-surgical risk in any manner. For example, the display unit 73 does not need to display the graph 73a and the predicted value 73b on the same screen. The display unit 73 may switchably display the graph 73a and the predicted value 73b . The display unit 73 may display a switching button and switch the display in response to the doctor pressing the button.

In the display unit 73, the area of the graph 73a may be different from the area of the predicted value 73b . For example, in the example of FIG. 6, the graph 73a and the predicted value 73b are illustrated with similar areas, but one may be displayed larger than the other. For example, the display unit 73 may be able to adjust the display regions of the graph 73a and the predicted value 73b according to a screen size (for example, numerical value input or drag operation) designated by the doctor.

Furthermore, the display unit 73 may arrange the graph 73a and the predicted value 73b not only on the left and right parts but also on the upper and lower parts. For example, in a case where a period indicated by the graph 73a is long, a horizontally long layout is suitable. In this case, the display unit 73 may adjust the arrangement with the predicted value 73b in such a way that the time axis of the graph 73a can be sufficiently secured. As a result, it is possible to improve the visibility of the graph 73a.

The display unit 73 may perform color coding in order to improve the visibility of the graph 73a and the predicted value 73b . For example, the display unit 73 may allow the doctor to intuitively ascertain the degree of risk by using the green color for a low risk, the yellow color for a medium risk, and the red color for a high risk. Furthermore, in a case where the specific index exceeds a reference value, the display unit 73 may perform display in a display form such as highlighting, blinking, or changing the background color.

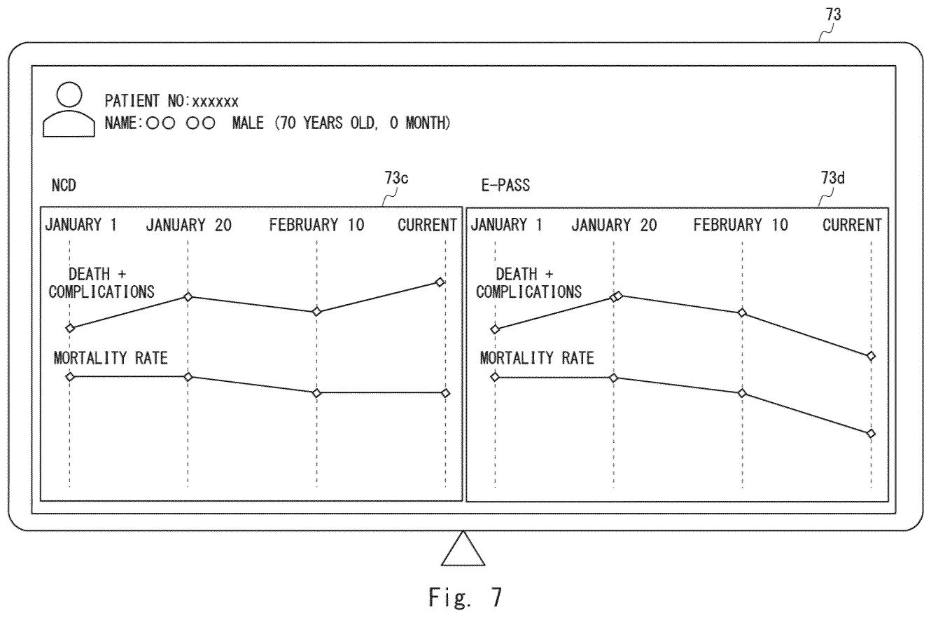

FIG. 7 is a view illustrating an example of a display screen for displaying the assessment result of the pre-surgical risk in a plurality of assessment indices. Graphs 73c and 73d illustrated in FIG. 7 are graphs similar to the graph 73a illustrated in FIG. 6. Unlike FIG. 6, in FIG. 7, the display unit 73 displays the graphs 73c and 73d relevant to two assessment indices. As described above, by displaying the graphs relevant to the plurality of assessment indices, the doctor can more easily make a determination on the pre-surgical risk assessment. Since the determination on the risk is facilitated, the doctor can perform risk assessment on many patients. As a result, this can lead to improvement in outcome and improvement in hospital bed rotation rate. The display unit 73 may display graphs relevant to three or more assessment indices.

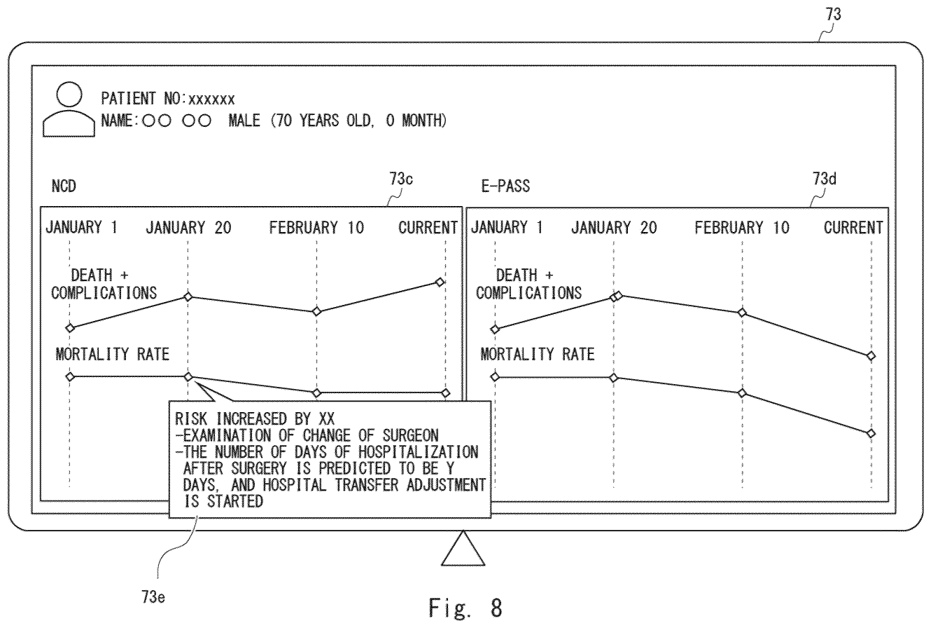

FIG. 8 is a view illustrating an example of a display screen for further displaying the proposal to the doctor. In the example of FIG. 8, the display unit 73 displays a comment 73e in addition to the graphs illustrated in FIG. 7. In this example, the comment 73e includes an increase in risk due to surgery, examination of a change of the surgeon, prediction of the number of days of hospitalization after surgery, and a proposal to start adjustment of hospital transfer. As described above, the comment 73e presents a policy according to the change in the pre-surgical risk, and thus the doctor can easily examine the response.

### (Processing Block E)

The description will return to FIG. 4. The processing block E is a block that detects insufficiency of patient data and performs processing of complementing the patient data, in a case where the patient data necessary for the pre-surgical risk assessment is insufficient. The processing block E functions as an insufficiency determination unit that determines whether necessary patient data is insufficient. In a case where it is determined that the necessary patient data is insufficient, the processing block E causes a display unit 72 to display a message indicating that the necessary patient data is insufficient.

The processing block E includes a necessary data collection unit 41, an insufficient data determination unit 51, the display unit 72, and an input unit 71. Since the necessary data collection unit 41 is as described above, the description of the necessary data collection unit 41 will be omitted here.

The insufficient data determination unit 51 determines whether the patient data necessary for the pre-surgical risk assessment is insufficient. The case where the necessary patient data is insufficient means, for example, a case where the smoking history is not described in an interview record due to forgetting to confirm the smoking history at the time of interview. The insufficient data determination unit 51 confirms whether the patient data collected by the necessary data collection unit 41 satisfies all pieces of data necessary for the pre-surgical risk assessment, and determines that the necessary patient data is insufficient in a case where the patient data does not satisfy all the pieces of data necessary for the pre-surgical risk assessment. In a case where it is determined that the necessary patient data is insufficient, the insufficient data determination unit 51 transmits information regarding the insufficient patient data to the display unit 72.

The display unit 72 displays the information regarding insufficient patient data. Similar to the display unit 73, the display unit 72 is a display device such as a display. The display unit 73 and the display unit 72 may be the same display device.

The input unit 71 is input means for a doctor or a medical worker who has confirmed the insufficient data displayed on the display unit 72 to input data. The input unit 71 is, for example, an input device such as a keyboard or a mouse. The input unit 71 may be a touch panel integrally formed with the display unit 72. For example, in a case where there is no description of the smoking history, the doctor or the medical worker confirms the patient by telephone or the like, and updates the record via the input unit 71. As a result, since the patient data is complemented, the pre-surgical risk can be assessed.

The configuration of the server 100 has been described above. The server 100 includes a processor, a memory, and a storage device as a configuration (not illustrated). The storage device stores a computer program in which the processing according to the present disclosure is implemented. The processor may cause the computer program to be read from the storage device into the memory, and may execute the computer program. As a result, the processor implements the functions of the data processing unit 11, the data type determination unit 13, the test numerical value/patient condition extraction unit 16, the data processing unit 21, the necessary input item extraction unit 23, the order input monitoring unit 31, the patient condition monitoring unit 32, the pre-surgical risk assessment start determination unit 33, the pre-surgical risk assessment index selection unit 34, the necessary data collection unit 41, the pre-surgical risk comparison unit 43, and the insufficient data determination unit 51 (referred to as the “data processing unit 11 and the like” below).

Alternatively, each of the data processing unit 11 and the like may be achieved by dedicated hardware. Some or all of the constituent components may be achieved by general-purpose or dedicated circuitry, a processor, or the like, or a combination thereof. These may be configured by a single chip, or may be configured by a plurality of chips connected via a bus. Some or all of the constituent components may be achieved by a combination of the above-described circuitries or the like and the program.

### [External Server 200]

The external server 200 includes the pre-surgical risk calculation unit 210. As described above, the pre-surgical risk calculation unit 210 performs the pre-surgical risk assessment in response to a request from the necessary data collection unit 41 of the server 100. The pre-surgical risk calculation unit 210 performs the pre-surgical risk assessment by using the patient data collected by the necessary data collection unit 41 and the assessment index stored in the pre-surgical risk assessment index storage unit 22, and sends the assessment result to the pre-surgical risk storage unit 42.

The external server 200 includes a processor, a memory, and a storage device as a configuration (not illustrated). The storage device stores a computer program in which the processing according to the present disclosure is implemented. The processor may cause the computer program to be read from the storage device into the memory, and may execute the computer program. As a result, the processor implements the function of the pre-surgical risk calculation unit 210.

The configuration of the information processing system 500 has been described above. The configurations of the server 100 and the external server 200 described above are merely examples, and can be appropriately changed. For example, in a case where some or all of the constituent components of the server 100 and the external server 200 are achieved by a plurality of information processing apparatuses, circuitries, and the like, the plurality of information processing apparatuses, circuitries, and the like may be arranged in a centralized manner or in a distributed manner. For example, each of the information processing apparatuses, circuitries, and the like may be achieved in the form of a client server system, a cloud computing system, or the like in which they are connected to each other through a communication network. For example, the functions of the server 100 and the external server 200 may be provided in a software as a service (SaaS) format.

The server 100 and the external server 200 may use AI in the above-described various types of processing.

### (Processing of Server 100)

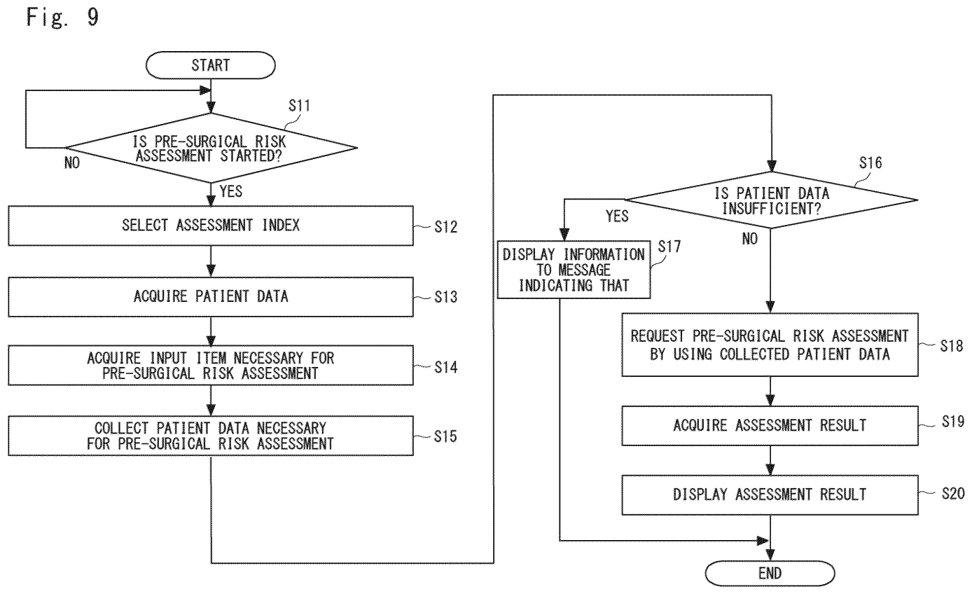

Subsequently, processing performed by the server 100 will be described with reference to FIG. 9. FIG. 9 is a flowchart illustrating a flow of processing performed by the server 100. It is assumed that, in the processing blocks A and B illustrated in FIG. 4, processing is normally performed, and necessary data is already stored in the test numerical value/patient condition storage unit 17 and the necessary input item storage unit 24. Therefore, detailed flowcharts in the processing blocks A and B are not illustrated.

First, the pre-surgical risk assessment start determination unit 33 determines whether to start pre-surgical risk assessment based on a monitoring result of the order input monitoring unit 31 or the patient condition monitoring unit 32 (S11). In a case where it is determined not to start the pre-surgical risk assessment (NO in S11), the process of Step S11 is repeated. In a case where it is determined to start the pre-surgical risk assessment (YES in S11), the pre-surgical risk assessment index selection unit 34 selects an assessment index relevant to surgery of a patient (S12).

The necessary data collection unit 41 acquires patient data from the test numerical value/patient condition storage unit 17 (S13). Subsequently, the necessary data collection unit 41 acquires an input item necessary for the pre-surgical risk assessment from the necessary input item storage unit 24 (S14). The necessary data collection unit 41 collects patient data necessary for risk assessment based on the necessary input item and the patient data (S15). For example, the necessary data collection unit 41 refers to the correspondence table as illustrated in FIG. 5 and collects the necessary patient data.

The insufficient data determination unit 51 determines whether the necessary patient data is insufficient (S16). In a case where it is determined that the necessary patient data is insufficient (YES in S16), the display unit 72 displays a message indicating that the necessary patient data is insufficient (S17). In a case where it is determined that the necessary patient data is not insufficient (NO in S16), the necessary data collection unit 41 requests the external server 200 to perform pre-surgical risk assessment by using the collected patient data (S18).

The pre-surgical risk comparison unit 43 acquires an assessment result via the pre-surgical risk storage unit 42 (S19). The pre-surgical risk comparison unit 43 compares the pre-surgical risk and stores a comparison result in the pre-surgical risk comparison result storage unit 44. The display unit 73 displays the assessment result (S20). As a result, for example, the display unit 73 displays the assessment results as illustrated in FIGS. 6 to 8, and ends the processing.

As described above, in the information processing system 500 according to the present disclosure, the server 100 collects patient data necessary for risk assessment based on necessary input items and patient data. As a result, it is possible to easily collect data necessary for risk assessment of surgery.

As a result, it is possible to easily perform pre-surgical risk assessment, and thus, the information processing system 500 can achieve a change of a treatment policy, improvement of a post-surgical outcome, and improvement of a hospital bed rotation rate. According to the information processing system 500, it is possible to achieve pre-surgical risk assessment for all patients. The information processing system 500 can achieve more precise and accurate risk assessment by performing time-series comparison of assessment indices and comparison of a plurality of assessment indices.

### Modified Example

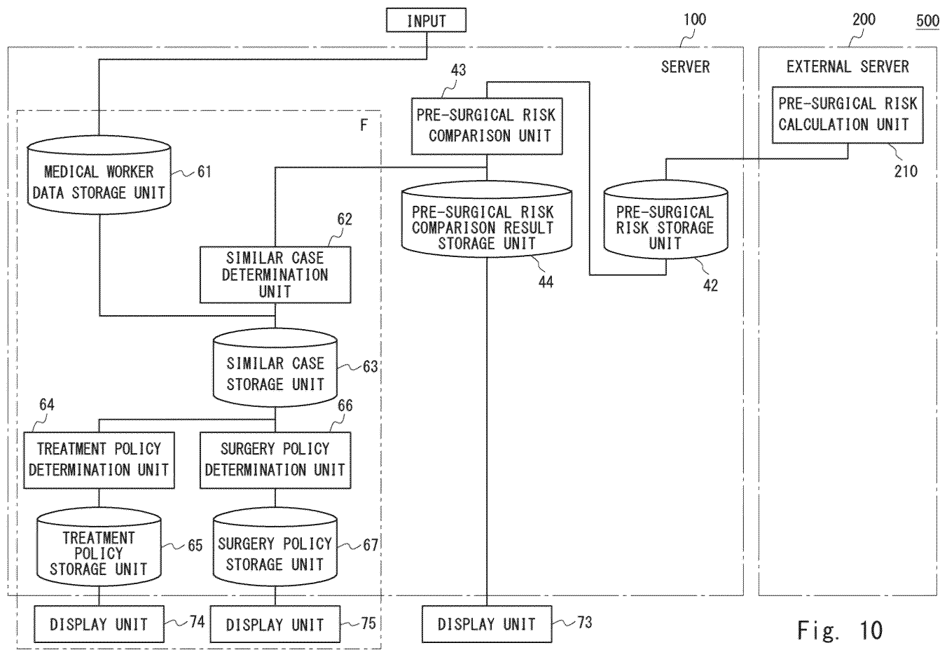

FIG. 10 is a block diagram illustrating a configuration of a processing block F according to a modified example of the second example embodiment. As illustrated in FIG. 10, the server 100 described above may further include a processing block F. In FIG. 10, some of the functional units described above are omitted.

### (Processing Block F)

The processing block F is a block that determines a surgery policy and a treatment policy suitable for a patient with reference to a past similar case. The processing block F functions as a policy determination unit that determines a policy for the surgery or a policy for a post-surgery treatment based on whether there is a case similar to the case of the patient. The processing block F includes a medical worker data storage unit 61, a similar case determination unit 62, a similar case storage unit 63, a treatment policy determination unit 64, a treatment policy storage unit 65, a surgery policy determination unit 66, and a surgery policy storage unit 67.

The medical worker data storage unit 61 stores information of a medical worker involved in surgery and treatment. For example, in the case of a doctor, the medical worker data storage unit 61 may include information such as service years, presence or absence of qualification as a specialist, the number of times of performing a specific surgical method (for example, surgical method A), and the like.

The similar case determination unit 62 refers to a past similar case and determines similarity with a current patient case. The similar case determination unit 62 sends a determination result to the similar case storage unit 63. The similar case storage unit 63 stores the determination result of the similar case determination unit 62. For example, the similar case storage unit 63 stores the current patient case, the past similar case, and the similarity in association with each other.

The treatment policy determination unit 64 and the surgery policy determination unit 66 respectively determine a treatment policy and a surgery policy suitable for the current patient based on information of the similar case. The treatment policy determination unit 64 and the surgery policy determination unit 66 send determination results to the treatment policy storage unit 65 and the surgery policy storage unit 67, respectively. The treatment policy storage unit 65 and the surgery policy storage unit 67 store a treatment policy and a surgery policy, respectively.

A display unit 74 and a display unit 75 display information stored in the treatment policy storage unit 65 and the surgery policy storage unit 67, respectively. The display unit 74 and the display unit 75 may have configurations equivalent to those of the display unit 72 and the display unit 73 described above.

With such a configuration, in the present modified example, it is possible to support determination of more appropriate surgery policy and treatment policy by utilizing past case data and the information of a medical worker.

### Configuration Example of Hardware

Each functional configuration unit of the information processing apparatus 1, the server 100, and the external server 200 (referred to as “information processing apparatus 1 and the like” below) may be achieved by hardware (for example, a hard-wired electronic circuit or the like) that implements each functional component, or may be achieved by a combination of hardware and software (for example, a combination of an electronic circuit and a program that controls the electronic circuit, or the like). A case where each functional configuration unit of the information processing apparatus 1 and the like is achieved by a combination of hardware and software will be described below.

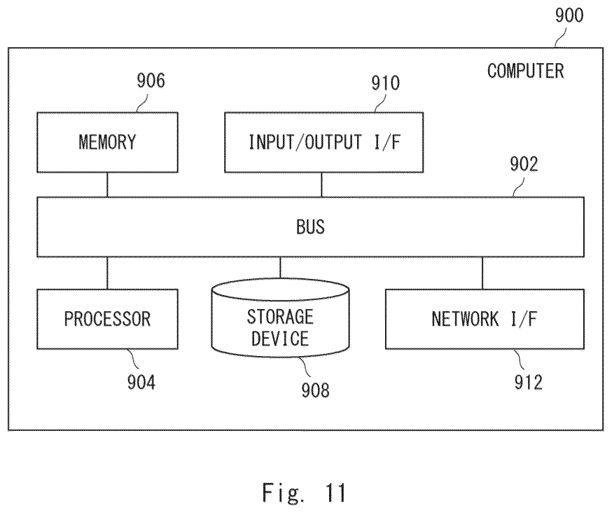

FIG. 11 is a block diagram exemplifying a hardware configuration of a computer 900 that implements the information processing apparatus 1 and the like. The computer 900 may be a dedicated computer designed to implement the information processing apparatus 1 and the like, or may be a general-purpose computer. The computer 900 may be a portable computer, such as a smartphone, a tablet terminal, or the like.

For example, by installing a predetermined application in the computer 900, each function of the information processing apparatus 1 and the like is achieved in the computer 900. The application mentioned above includes programs for implementing the functional configuration units of the information processing apparatus 1 and the like.

The computer 900 includes a bus 902, a processor 904, a memory 906, a storage device 908, an input/output interface 910, and a network interface 912. The bus 902 is a data transmission path through which the processor 904, the memory 906, the storage device 908, the input/output interface 910, and the network interface 912 mutually exchange data. However, a method for connecting the processor 904 and the like to each other is not limited to the bus connection.

The processor 904 is various processors such as a central processing unit (CPU), a graphics processing unit (GPU), a field-programmable gate array (FPGA), and a quantum processor (quantum computer control chip). The memory 906 is a main storage device achieved using a random access memory (RAM) or the like. The storage device 908 is an auxiliary storage device achieved by using a hard disk, a solid state drive (SSD), a memory card, read only memory (ROM), or the like.

The input/output interface 910 is an interface for connecting the computer 900 with an input/output device. For example, an input device such as a keyboard and an output device such as a display device are connected with the input/output interface 910.

The network interface 912 is an interface for connecting the computer 900 to a network. The network may be a local area network (LAN) or a wide area network (WAN).

The storage device 908 stores the programs for implementing the functional configuration units of the information processing apparatus 1 and the like (programs for implementing the application mentioned above). The processor 904 reads the programs into the memory 906 and executes them, thereby implementing the functional configuration units of the information processing apparatus 1 and the like.

Each of the processors executes one or more programs including instructions for causing a computer to perform an algorithm. The program includes instructions (or software codes) for causing the computer to implement one or more functions described in the example embodiments in a case of being read by the computer. The program may be stored in various types of non-transitory computer-readable media or tangible storage media. By way of example, and not limitation, non-transitory computer-readable media or tangible storage media include RAM, ROM, flash memory, SSD or other memory technology, CD-ROM, digital versatile disc (DVD), Blu-ray® disk or other optical disk storage, magnetic cassettes, magnetic tape, magnetic disk storage, or other magnetic storage devices. The program may also be transmitted in the various types of transitory computer-readable media or a communication medium. Without limitation, examples of the transitory computer readable medium or the communication medium include an electric signal, an optical signal, an acoustic signal, or any other form of propagation signal.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with at least one of embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

The present disclosure is not limited to the example embodiments described above, and may be appropriately changed without departing from the gist. For example, in the above description, surgery is used as a target of risk assessment, but the present disclosure can also be applied to risks other than surgery (for example, risks of various procedures). The above-described example embodiments can be executed in any combination. For example, the first example embodiment, the second example embodiment, and the modified example may be executed in any combination.

Some or all of the above-described example embodiments may also be described as the following Supplementary Notes, but are not limited to the following Supplementary Notes.

### (Supplementary Note 1)

An information processing apparatus including:
a patient data acquisition unit that acquires patient data that is extracted from an electronic medical record and indicates a condition of a patient;
a necessary input item acquisition unit that acquires an input item necessary for risk assessment for surgery of the patient;
a necessary data collection unit that collects patient data necessary for the risk assessment based on the necessary input item and the patient data; and
a risk assessment request unit that requests the risk assessment by using the collected patient data.

### (Supplementary Note 2)

The information processing apparatus according to Supplementary Note 1, further including an electronic medical record data organization unit that organizes electronic medical record data included in the electronic medical record for use in the risk assessment,
in which the patient data acquisition unit acquires patient data included in the organized electronic medical record data.

### (Supplementary Note 3)

The information processing apparatus according to Supplementary Note 2, in which the electronic medical record data organization unit determines whether the electronic medical record data is directly available for the risk assessment based on a data type of the electronic medical record data, and performs predetermined processing in a case where it is determined that the electronic medical record data is not directly available.

### (Supplementary Note 4)

The information processing apparatus according to Supplementary Note 1 or 2, further including a monitoring unit that monitors at least one of an order status of surgery to be performed on the patient and a condition of the patient, and determines whether to perform the risk assessment based on a monitoring result,
in which the monitoring unit determines to perform the risk assessment in at least one of a case where surgery of the patient is ordered and a case where an abnormality is detected in the condition of the patient.

### (Supplementary Note 5)

The information processing apparatus according to Supplementary Note 4, in which the necessary data collection unit starts collection of the patient data necessary for the risk assessment in a case where the monitoring unit determines to perform the risk assessment.

### (Supplementary Note 6)

The information processing apparatus according to any one of Supplementary Notes 1 to 5, further including a necessary input item management unit that manages the necessary input items based on an assessment index set in advance as a reference of the risk assessment,
in which the necessary input item acquisition unit acquires the necessary input item based on the assessment index.

### (Supplementary Note 7)

The information processing apparatus according to any one of Supplementary Notes 1 to 6, in which the necessary data collection unit collects the necessary patient data based on a predetermined correspondence table in which the necessary input item is associated with the patient data including a description relevant to the necessary input item.

### (Supplementary Note 8)

The information processing apparatus according to any one of Supplementary Notes 1 to 7, further including a display unit that displays a result of the risk assessment, in which the display unit displays a predicted value of a post-surgical outcome indicating a magnitude of a risk in the surgery.

### (Supplementary Note 9)

The information processing apparatus according to Supplementary Note 8, in which the display unit displays the result at a plurality of assessment timings as time-series data.

### (Supplementary Note 10)

The information processing apparatus according to Supplementary Note 8 or 9, in which the display unit displays the result calculated based on the same assessment index set in advance, by using the predicted value and a time-series graph of the predicted value.

### (Supplementary Note 11)

The information processing apparatus according to any one of Supplementary Notes 8 to 10, in which the display unit displays each of a plurality of the results calculated based on a plurality of assessment indices set in advance on the same screen.

### (Supplementary Note 12)

The information processing apparatus according to any one of Supplementary Notes 8 to 11, in which the display unit further displays, on the same screen, a comment including a proposal related to a policy for the surgery, the proposal being created based on the result.

### (Supplementary Note 13)

The information processing apparatus according to any one of Supplementary Notes 1to 12, further including an insufficiency determination unit that determines whether the necessary patient data is insufficient,
in which in a case where it is determined that the necessary patient data is insufficient, the insufficiency determination unit causes a display unit to display a message indicating that the necessary patient data is insufficient.

### (Supplementary Note 14)

The information processing apparatus according to any one of Supplementary Notes 1 to 13, further including a policy determination unit that determines a policy for the surgery or a policy for a post-surgery treatment based on whether there is a case similar to a case of the patient.

### (Supplementary Note 15)

An information processing method including:
a patient data acquisition step for acquiring patient data that is extracted from an electronic medical record and indicates a condition of a patient;
a necessary input item acquisition step for acquiring an input item necessary for risk assessment for surgery of the patient;
a necessary data collection step for collecting patient data necessary for the risk assessment based on the necessary input item and the patient data; and
a risk assessment request step for requesting the risk assessment by using the collected patient data.

### (Supplementary Note 16)

A program for causing a computer to execute:
a patient data acquisition step for acquiring patient data that is extracted from an electronic medical record and indicates a condition of a patient;
a necessary input item acquisition step for acquiring an input item necessary for risk assessment for surgery of the patient;
a necessary data collection step for collecting patient data necessary for the risk assessment based on the necessary input item and the patient data; and
a risk assessment request step for requesting the risk assessment by using the collected patient data.

Some or all of the elements (for example, configurations and functions) that have been described in Supplementary Notes 2 to 14, which are dependent on Supplementary Note 1, may depend from Supplementary Notes 15 and 16 as well with dependency relationships similar to those of Supplementary Notes 2 to 14. Some or all of the elements that have been described in any Supplementary Note are applicable to various types of hardware, software, recording means for recording software, systems, and methods.

What is claimed is:

1. An information processing apparatus comprising:
at least one memory storing instructions; and
at least one processor configured to execute the instructions to:
acquire patient data that is extracted from an electronic medical record and indicates a condition of a patient;
acquire an input item necessary for risk assessment for surgery of the patient;
collect patient data necessary for the risk assessment based on the necessary input item and the patient data;
request the risk assessment by using the collected patient data;
organize electronic medical record data included in the electronic medical record for use in the risk assessment;
acquire patient data included in the organized electronic medical record data; and
determine whether the electronic medical record data is directly available for the risk assessment based on a data type of the electronic medical record data, and perform a predetermined process in a case of determining that the electronic medical record data is not directly available.

2. The information processing apparatus according to claim 1, wherein the at least one processor is further configured to execute the instructions to
monitor at least one of an order status of surgery to be performed on the patient and a condition of the patient, and determine whether to perform the risk assessment based on a monitoring result, and
determine to perform the risk assessment in at least one of a case where surgery of the patient is ordered and a case where an abnormality is detected in the condition of the patient.

3. The information processing apparatus according to claim 2, wherein the at least one processor is further configured to execute the instructions to start collection of the patient data necessary for the risk assessment in a case where the risk assessment is determined to be performed.

4. The information processing apparatus according to claim 1, wherein the at least one processor is further configured to execute the instructions to
manage the necessary input item based on an assessment index set in advance as a reference of the risk assessment, and
acquire the necessary input item based on the assessment index.

5. The information processing apparatus according to claim 1, wherein the at least one processor is further configured to execute the instructions to collect the necessary patient data based on a predetermined correspondence table in which the necessary input item is associated with the patient data including a description relevant to the necessary input item.

6. The information processing apparatus according to claim 1, wherein the at least one processor is further configured to execute the instructions to
display a result of the risk assessment on a display unit, and
display a predicted value of a post-surgical outcome indicating a magnitude of a risk in the surgery.

7. The information processing apparatus according to claim 6, wherein the at least one processor is further configured to execute the instructions to display the result at a plurality of assessment timings as time-series data.

8. The information processing apparatus according to claim 6, wherein the at least one processor is further configured to execute the instructions to display the result calculated based on the same assessment index set in advance, by using the predicted value and a time-series graph of the predicted value.

9. The information processing apparatus according to claim 6, wherein the at least one processor is further configured to execute the instructions to display each of a plurality of the results calculated based on a plurality of assessment indices set in advance on the same screen.

10. The information processing apparatus according to claim 6, wherein the at least one processor is further configured to execute the instructions to further display, on the same screen, a comment including a proposal related to a policy for the surgery, the proposal being created based on the result.

11. The information processing apparatus according to claim 1, wherein the at least one processor is further configured to execute the instructions to
determine whether the necessary patient data is insufficient, and
display a message indicating that the necessary patient data is insufficient on a display unit in a case where it is determined that the necessary patient data is insufficient.

12. The information processing apparatus according to claim 1, wherein the at least one processor is further configured to execute the instructions to determine a policy for the surgery or a policy for a post-surgery treatment based on whether there is a case similar to a case of the patient.

13. An information processing method comprising:
acquiring patient data that is extracted from an electronic medical record and indicates a condition of a patient;
acquiring an input item necessary for risk assessment for surgery of the patient;
collecting patient data necessary for the risk assessment based on the necessary input item and the patient data;
requesting the risk assessment by using the collected patient data;
organizing electronic medical record data included in the electronic medical record for use in the risk assessment;
acquiring patient data included in the organized electronic medical record data; and
determining whether the electronic medical record data is directly available for the risk assessment based on a data type of the electronic medical record data, and performing a predetermined process in a case of determining that the electronic medical record data is not directly available.

14. A non-transitory computer readable medium storing a program for causing a computer to execute:
acquiring patient data that is extracted from an electronic medical record and indicates a condition of a patient;
acquiring an input item necessary for risk assessment for surgery of the patient;
collecting patient data necessary for the risk assessment based on the necessary input item and the patient data;
requesting the risk assessment by using the collected patient data;
organizing electronic medical record data included in the electronic medical record for use in the risk assessment;
acquiring patient data included in the organized electronic medical record data; and
determining whether the electronic medical record data is directly available for the risk assessment based on a data type of the electronic medical record data, and performing a predetermined process in a case of determining that the electronic medical record data is not directly available.
